(19) United States
(12) Patent Application Publication
(10) Pub. No.: US 2008/0033564 A1
(43) Pub. Date: Feb. 7, 2008

(54) Artificial Joint

(75) Inventors: Dietmar Kubein-Meesenburg, Kreiensen OT Greene (DE); Hans Naegerl, Gleichen (DE); Joachim Theusner, Muenchen (DE)

Correspondence Address: DARBY & DARBY P.C., P.O. BOX 770, Church Street Station, New York, NY, 10008-0770, US

(73) Assignee: H J S GELENK SYSTEM GMBH, Muenchen (DE)

(21) Appl. No.: 10/572,081

(22) PCT Filed: Aug. 26, 2004
(86) PCT No.: PCT/DE04/01888

§ 371 Date: Feb. 20, 2007

(30) Foreign Application Priority Data
Sep. 19, 2003 (DE) 103 43 889.0

(51) Int. Cl. A61F 2/30

(52) U.S. Cl. 623/18.11

(57) ABSTRACT

An artifieial joint includes a condyle and a jointzsocket having a socket eleinent and an inlay whose associated functional surfaces functionally interlinked. The functional surface facing the joint socket has circular, concave section contours. the orbital radii of the joint deviating fromn each other in a main fltnetional plane relative to a secondary fuinctional plane. The functional surface can be locked in various positions 5relative to the joint socket and, the joint inctides snap-in stages, produced by gear cutting, between the joint element and the inlav with the flinctional surface, which allonw for locking the position in steps of I

The invention relates to an artificial joint, etspecially to replace a humnan hip joint, comprising a condyle and a joint socket wl-iose associated fuictional surfaces are functionally interlinked.

### BACKGROUND

Such an artificial condyle for use in a human hip joint is known. for examt-ple, from EP 04 63 011 B1. This :specificanion describes an artificial joint consisting of at least two joint parts with sphierical functional surfaces that move towards each other. The curvature relationships of the fuinictional surfaces having a circular section contour are convex-convex, convex-concave or concave-concave relative to ea h other and the joint geometry is determined by a joint chain having two joint axes that run through the rotation centers M1 and M2 of the functional surfaces having the radii RI and R2. In order to create a pressure-stable joint system and thus to ensure a natural function that is comfortable for humans, a functional element havina a specific geometry is located betveen the two functional surfaces. The parallel or serial arrangement of such a joillt is likewise desecibed.

Moreover, EP 09 69 781 B1 has already described an artificialajoint especially for replacing a hruman h1ip joint. The artificial condyle is fitted with a spherically shaped joint socket whose crorss section has a circular, concave section contotir and, vhich has a curvature radius R1 and a curvature miidpoint M1. At least in its area that articulates in the joint socket, a spherically convex functional suraace is Xformed in such a way that, when said surface is in the state of having been inserted into the sockct, a radius RK1=R1 with a midpoint MK1 is created in a lengthwise plane X-X running tihrough the maidpoint M1, said midpoint MK1 coinciding with M1. In this manner, a pressure distribution element is not needed and, at the isame time. a pressure-stable turned-over dimerous chain is realized that is always the same size when the main functional plane is pivoted.

Other artificial joints are also lnown from EP 06 17 595 B, EP 07 34 701 B, EP 06 91 830 B as well as EP 09 31 758 B.

### SUMMARY OF THE INVENTION

An object of the present invention is to provide an artificial joint that has a condyle and a joint socket and that is intended to replace a human hip joint, in suchla way that the load-bearing capacity of tlhe joint as well as the comifort of the patient are considerably improved.

Hence, according to the invention, an artificial joint is provided with which, for pubposes of individually adapting the artificial joint to the patient, at least one of the functional surfaces can be affixed in differeint positions relative to the: associated joint socket or condyle. The orbital radii of each of the associated functional stirfaces in the main functional plane and in the secondary finctional plane that is rotated by 90° with respect to the main functional plane can be optimally alilgned with the frontal plane as well as with the sagittal plane of the patient. In particular, interacting forces acting in the frontal plane are riiot transntitted to the connection between the joint socket and the bone. Rather, the divergent design of the orbital radii of each of the associated functional surfaces allows a deflection movement by ineans of which the transmission of ham-iful forces is ruled: out.

Fundamentally, the difference between the orbital radii of the main functional plane and of the secondary functional plane can be achieved by an appropriate design of the first and second ftrnctional surlaces. IIn contrast, it is especially advantageous if a first functional surface displays ani asymimetry in the main functional plane as compared to the seconidary functional plane while the seconcd functional suirfice exhlibits symtimetrical functional planes. In this marrier, only one of the two flinctional surfaces has to have. a concave or convex section contour that differs fi-om a spherical shape, as a result of which the production process is facilitated.

It has proven to be especially advantageous if tlhe first finctional surface is associated with tlhe joint socket and the second fuinctional surface is associated w,itlh tlhe condyle. As a result, it is -a,voided that a rotation of the condyle interferes with this deflection movement, thus preventing undesired high stress on the connection between the joint socket and the bone. In particular, this prevents that a rotation of the condyle rmight be able to shift the finctional surface with the: smaller orbital radius out of the frontal plane,

According to an especially advantageous embodiment, the first fiunctional surface is designed so as to be drit-shiaped or spindleshaped or oval, so that the desired joint geometry can be realized Din a simple maimer.

After the appertaining joint socket has been affixed to the bone, the functional surface could first be positioned relative to the bone and then affixed permanently in the position thus established by means of an adhesive connection. In contrast, according to an especially practical embodiment, in order to set different positions, the flictional surface can be affixed in different locking stages relative to thle associated joint socket or to the condyle. Tihis is aechieved, for example, by teeth located between the joint socket and the fimetioncal surface that allow the fixation of the relative position in 0.50 to 10″ increments. This relatively easy adjustability can shorten the duration of the surgery considerably.

According to another especially advantageous modification, the flnctional surface can be secured in different positions relative to the associated joint socket or condyle by means of a positive connection. Consequently, the load-bearing capacitv of the connection thus created is additionally increased. Moreover, a precise positioning is made possible in this mianner.

The functional surface could be non-detachably locked by, being inserted into the joint socket. However, an especially tadvantageous embodiment of the present invention is one in which the functional surface can be affixed in different positiolns relative to the associated joint socket or condyle bly means of a shrinkage connection. This achieves a nion-positive connection that can be positioned with little effort and that is also highly stressable as well as optionally a positive connection that is non-detachable at body temperature.

An especially practical modification is also achieved in that the diameter of the functional surface of the condyle or of the joint socket in the frontal plane of the patient is to be dimensioned betnveeln 0,5 mm and 8 mm, especially 2 mm, greater than the diameter of the functional surface of the conidyle or the diameter of the functional surface of the joint socket in the sagittal plane, so that, in this rnannier, thie desired deflection movement in the frontal plane is ensured and, at the same time, the stability of the joint is not considerably reduced. In the sagittal plane, the diameter of the functional surface 5 in comparison to the diameter of the functional surface 4 is to be provided with a play of 0.2 mm to 2 mm. In addition, the functional surface of the condyle can be non-spherical in shape, whereby this functional surface in the sagittal plane has to have the largest radiius.

Suitable materials are fundamentally all materials that are commonly employed in medical teclnology, whereby especially one embodiment in which the functional surfaces are made of polyethylene allows particularly simple handling,

### BRIEF DESCRIPTION OF THE DRAWINGS

The invention allows various embodiments. In order to fuirther illustrate their basic principle, one of them is shown in the dranving and is described below. The following is shown:

FIG. 1 an artificial joint in a frontal section,

FIG. 2 the artificial joint of FIG. 1 in a sagittal section; and

FIG. 3 the artificial joinit shown in FIGS. 1 and 2 in a cutaway top view.

### DETAILED DESCRIPTION OF A PREFERRED EMBODIMENT

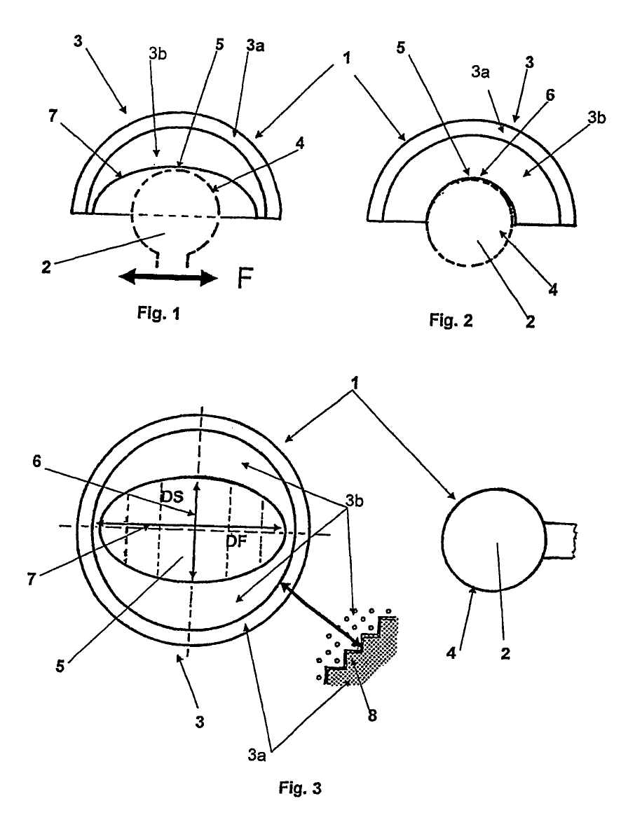

The fundamental stiicture of an artificial joint 1 accordinpg to the inivention, especially one that is intended to replace a hu-man hiip joint. is described with reference to FIGS. 1 and 2, where a cutaway depiction shows the joint 1 in a plane running parallel to the front as well as in a sagittal plane perpendicular thereto. The joinit 1 has a spherical condyle 2 and a joint soclket 3, consisting of a socket part 3a and an inlay 3b, whose associated finctional surfaces 4, 5 are functionally interlinlced. The cross section of the inlay 3b of thejoint socket 3 has concave section contours. whereby the orbital radii 6, 7 of the fuinctional surface 5 of the inlay 3b differ from each other in a main functional plane relative to the secondary furctional plane that is rotated by 90° with respect to the tmain functional plane. Therefore, tlhis configuration of the orbital radii 6, 7 of the functional surface 5 allows a deflection movement by means of which an acting external force F does not cause damage to the connection betweenl the joint socket 3 and la bone (not shown here).

FIG. 3 shows the antificial joint 1 depicted in FIGS. 1 and 2 additionally in a cutaway top view as well as in an enlarged detail view of the joint socket 3 with the socket part 3a and the inlay 3b as well as the condyle 2 includding the: fmcntional surfaces 4, 5. The diarneter DF of the functional surface 5 of the joint socket 3 in thie frontal plane is approxii-iiately 2 mm greater than the diameter DS in the sagittal plane, thus yielding the additional degree of freedom. In order for the orbital radii 6, 7 determined by the different diameters DF, DS of the ftinctioonal surface 5 in the main functional plane and in the secondary functional plane to be optinally aligned with the frontal plane as well as with the sagittal plane of the patient, the functional surface 5 can be affixed in different positions relative to the socket part 3a. This is done, for example, by locking stages 8 formed by teeth located between the socket pant 3a of the joint socket 3 and the inlay 3b with the functional surface 5, which allows a fixation of the relative position in 1° increments.

1-9. (canceled)

10: An artificial joint, comprising:
a condyle having a first functional surface; and
a joint socket haviIng including a socket part and an inlay having a second functional surface, the first and second functional surfaces being functionally interlinked, wherein at least one of the first and second finctional surface is non-splherical in shape, and eaclh of the first and second functional surfaces have orbital radii thai differ from each other in a main functional plane relative to a secondary fiunctional plane rotated by 900 with respect to the main functional plane, and wherein at least one of the functional surfaces is affixable in different positions relative to die respective joint socket or condyle so as to individually adapt the artificial joint to the patient.

11: The joint as recited in claim 10, wiherein one of the first and second functional surfaces displays an asymmetry in the main functional plane as compared to the secondary functional plane while the other of the first and second functionial surfaces :exhibits symnetrical functional planes.

12: The joi11t as recited in claim 10, wherein both the first and second functional surfaces are nioni-splherical.

13: The joint as recited in claim 10, wherein the first functional surface is one of drum-shaped, spindle-shaped, and oval.

14: The joint as recited in claim 10, wherein the first and second functional surfaces are affixable in different locking stages relative to the respective joinlt socket or condyLe so as to set the. different positions

15: The joint as recited in claimr 10, wherein one of the first and secondt fnictional surfaces is securable in the different positions relative to the respective joint socket or condyle by a positive coonnection.

16: The joifnt as recited in claim 1O, wherein one of the first and second functional surfaces is securable in the different positions relative to the respeentive joint socket or condyle by a slriiikage connectioni.

17: The joint as recited in claiim 10, wherein a diameter one ofttie first and second ftinctional surfaces in a frontal plane of the patient is between 0.5 mm and 8 mm greater than a diameter of the respective functional surface in a sagintal plane.

18: The joint as recited in clfaim 17, wherein the diameter one of the first anild second fiinctional surfaces in the frontal plane of the patient is 2 mm greater than the diameter of the respective functional surface in the sagittal plane.

19: The joint as recited in claim 10, wlerein the first and second flinctional surfacees are made ofpolyethylenes.

20: The joint as recited in claim 10, wherein the joint is a human hip replacement joint.
